(19) United States
(12) Patent Application Publication
(10) Pub. No.: US 2006/0217802 A1
(43) Pub. Date: Sep. 28, 2006

(54) Heart valve and method for insertion of the heart valve into a bodily vessel

(76) Inventors: Carlos Ruiz, New York, NY (US); Gerald Dorros, Wilson, WY (US)

Correspondence Address: KENYON & KENYON LLP, ONE BROADWAY, NEW YORK, NY, 10004, US

(21) Appl. No.: 11/303,761

(22) Filed: Dec. 15, 2005

Related U.S. Application Data
(60) Provisional application No. 60/593,173, filed on Dec. 16, 2004.

(51) Int. Cl. A61F 2/24

(52) U.S. Cl. 623/2.11; 623/2.18

(57) ABSTRACT

A heart valve includes a stent for fixing the heart valve in a bodily vessel and a stentless valve portion for restricting the flow of blood through the valve to a single direction. The valve portion is supported and suspended in the bodily vessel via connectors spanning a gap between the valve portion and the stent or by a direct end-to-end connection with the anchor portion.

### INCORPORATION BY REFERENCE

U.S. Provisional Patent Application No. 60/593,173, filed on Dec. 16, 2004 and entitled “Prosthetic Valve,” is expressly incorporated herein in its entirety by reference thereto. U.S. patent application Ser. No. ______ , entitled “A Separable Sheath and Method for Insertion of a Medical Device into a Bodily Vessel Using a Separable Sheath,” bearing Attorney Docket No. 13430/3, filed in the United States Patent and Trademark Office on the even date herewith, is also expressly incorporated herein in its entirety by reference thereto.

### FIELD OF THE INVENTION

The present invention relates to a heart valve and a method for its insertion into a bodily vessel.

### BACKGROUND INFORMATION

Valves, mechanical or biological, such as heart valves, can be implanted into a bodily vessel, for example, to replace native valves exhibiting abnormal anatomy and/or function as a result of congenital or acquired disease. Available prosthesis include two categories of valves including mechanical and biological. Mechanical valves include, for example, caged-ball valves, bi-leaflet valves or tilting disk valves. Biological valves are of two types: homografts, which are harvested from human cadavers, and biological tissue valves such as porcine aortic valves, pericardial sac tissue valves (porcine, equine, etc.), and other biological tissue valves, such as small intestinal submucosa tissue valves. These valves may be attached to a rigid or slightly flexible anchor, such as a stent, which may be covered with cloth, such as DACRON®. The stent or anchor may be attached to a sewing ring for fixation to a patient's native tissue.

Artificial valves are primarily designed to enable flow between two chambers by opening and closing, as well as to assure ease of implantation. U.S. Pat. No. 6,736,846 to Cox, herein incorporated by reference in its entirety, discloses the use of a tubular starting material to replace a diseased valve, such as any of the four heart valves. Cox stressed the importance of using a tubular valve, which if appropriately sized and having suitable material characteristics, would closely function as did the native valve.

Artificial valves function by solely enabling blood flow between two chambers or compartments. However, this is only one of many functions that is performed physiologically and functionally, although not readily apparent by the novice, by the native valve. Stents, disposed within or over a valve, can provide structural support for the valve, as well as enable attachment of the valve in a bodily vessel. The stent, while not altering the mechanical properties of the valve, may significantly affect the mechanical and physiological properties of the heart. Thus, a valve with a stent may not function exactly like the native valve. However, a stentless valve may more similarly reflect the physiologic function of a native valve. Therefore, there is believed to be a need for a valve for implantation into a bodily vessel, surgically and/or percutaneously, which may be secured in that bodily vessel, to more closely resemble physiologically the native valve.

### SUMMARY

According to an example embodiment of the present invention, a valve for placement in a bodily vessel includes: an anchor portion; a valve portion spaced apart from the anchor portion; and at least one connector connecting the anchor portion and adapted to support the valve in the bodily vessel.

The valve portion and the anchor portion may be configured to be delivered into the bodily vessel in a low profile and to be expanded to a larger profile, and the anchor portion may be adapted to anchor the valve in place in the bodily vessel.

The anchor portion may be mechanically expandable (such as by a balloon inflation, a wrench, electrically, magnetically, etc.), self-expandable, and/or may be made from a shape-memory material, etc., and may be constructed from an absorbable or non-absorbable material. The connector may include a strut extending along substantially an entire length of the valve portion, either longitudinally and/or perpendicularly in a circumferential manner at the level of the valve.

The valve portion may be substantially tubular and may include a plurality of flaps configured to allow fluid to pass therethrough in only one direction.

The valve portion may be made from biological materials, such as (a) small intestine sub-mucosa, (b) large tubular vascular structure, (c) pericardial tissue, (d) fascia lata, or (e) nano-synthesized material, such as stretchable Nitinol. The valve portion may also be made from other biocompatible materials, such as ePTFE, silk, Elast-Eon™, etc.

The valve portion may be made of an invaginated tube. An inner wall of the invaginated tube may be incised in at least two locations to form the flaps or leaflets, which permit unidirectional blood flow.

The anchor portion may include a stent and may be tapered toward the valve portion, for example, in a cylindrical or truncated conical form.

The connector may have a C-shaped terminal end that is proximal to the anchor to support the radial expansion of the tissue valve.

The connector may include a T-shaped retainer securing the tubular tissue of the external portion of the invaginated tube to each connector.

The T-shaped retainer may be disposed within a slot in the connector, and the valve portion may be arranged between each T-shaped retainer and connector.

The valve portion may be created and secured to the connectors utilizing one or more of, for example, glue, rivets, suture, staples, etc.

The connector may be constructed as part of the anchor device or may be attached to the anchor, for example, utilizing one or more of a chemical or physical adherence technique, suture, staples, rivets, etc.

A portion of the connector in contact with the valve portion may be ribbed.

A portion of the connector in contact with the valve portion may include bores.

The connector may be of sufficient length to allow the anchor portion to fully expand while the valve portion remains in a low profile state.

The valve portion itself includes a stent or may be stentless.

According to an example embodiment of the present invention, a method for implanting a valve into a bodily vessel, the valve including an anchor portion, a valve portion spaced apart from the anchor portion and at least one connector strut connecting the anchor portion and the valve portion, includes: (a) passing a balloon catheter having first (proximal) and second (distal) balloons on a proximal end retrograde into the bodily vessel, for example, over a guide wire, with the anchor portion mounted on the distal balloon in a low profile configuration and the valve portion mounted on the proximal balloon in a low profile configuration; (b) inflating the proximal balloon to expand the valve portion to a larger profile; (c) deflating the proximal balloon, (d) inflating the distal balloon to expand the anchor portion to a larger profile; (e) deflating the distal balloon; and (f) withdrawing the catheter from the bodily vessel. The valve portion would be supported in the bodily vessel by the connector strut. Inflation of the proximal balloon allows for positional adjustments of the valve portion prior to anchoring. However, the distal balloon may also be inflated first so as to expand the anchor portion prior to expansion of the valve portion. The valve may be stentless.

According to an example embodiment of the present invention, a method for implanting a valve into a bodily vessel, the valve including a self-expandable anchor portion, and a balloon valve portion spaced apart from the anchor portion by, at least one connector strut, which joins the anchor portion and the valve portion, includes: (a) inserting a delivery system sheath retrograde, (b) withdrawal of a sheath of the delivery system so as to enable expansion of the valve, and (c) expanding the valve using a balloon catheter of the delivery system. The valve portion may be supported in the bodily vessel by the connector strut and may be stentless.

According to an example embodiment of the present invention, a method includes: deploying a valve in a bodily vessel, including arranging an anchor portion of the valve in the bodily vessel on one side of a branch leading into the bodily vessel and arranging a valve portion of the valve in the bodily vessel on a side of the branch opposite to the anchor portion, at least one connector of the valve connecting the anchor portion to the valve portion and spanning the branch, the connector arranged to permit fluid flow therethrough between the branch and the bodily vessel.

According to an example embodiment of the present invention, a valve for placement in a bodily vessel includes: a stentless valve portion and an anchor portion situated end-to-end with the valve portion. Both expanded components may be attached so as to form a cylindrical or ovoid structure, with the anchor portion being self-expanding so as to attach to the walls of the bodily vessel. The stentless valve may be directly adherent end-to-end to the anchor portion which thereby obviates the necessity for a connector, such as a strut attachment, between the anchor portion and the valve.

According to an example embodiment of the present invention, a valve for placement in a bodily vessel includes: a stentless valve portion; and an anchor portion including a main body portion and at least one connector portion extending beyond a proximal end of the main body portion, the valve portion connected to a proximal portion of each connector portion, the valve portion spaced apart from the main body portion of the anchor portion, the valve portion supported by the connector portions of the stent.

The valve portion and anchor portion may be self-expandable and/or expandable using a retractable device. For example, the valve portion and anchor portion may be expanded using a balloon on, for example, a balloon catheter, or expanded using a retractable self expanding stent or any other retractable expandable device capable of expanding the valve portion and/or anchor portion.

According to an example embodiment of the present invention, a method includes: a) inserting a guide wire into the femoral vein, inferior vena cava (IVC), right atrium (RA), left atrium (LA), and then through the left ventricle (LV), ascending and descending aorta, abdominal aorta, iliac artery, and exteriorizing the guide wire at the femoral artery; b) retrogradely passing an insertion sheath, e.g., a sheath splittable (capable of being divided, for example, circumferentially) into proximal and distal portions or a sheath having releasably connectable proximal and distal portions, with a valve loaded therein over the guide wire such that a distal end of the sheath remains exteriorized through the femoral artery; c) moving the valve into deployment position near the anatomical location of the native aortic valve, wherein, when a balloon catheter is used, an anchor portion of the valve device is disposed over a distal balloon and a stentless valve portion is disposed over a proximal balloon of the balloon catheter, and wherein a proximal end of the distal portion of the sheath is disposed over the anchor portion and a distal end of the proximal portion of the sheath is disposed over the valve portion of the valve device; d) withdrawing the proximal portion of the sheath from the patient via the femoral vein; e) inflating the proximal balloon of the balloon catheter so as to expand the valve portion of the valve device, the valve device now being fully deployed; f) deflating the proximal balloon of the balloon catheter, which enables the valve to be fully expanded and functional; and g) at least partially withdrawing the distal portion of the sheath through the femoral artery cannulation site (which may optionally include a sheath system) so as to expose the anchor portion, the valve portion remaining covered by the proximal portion of the sheath, the distal end of the sheath may extend beyond the end of the balloon catheter and may be tapered to a size which allows free passage and movement over the guide wire; h) inflating the distal balloon so as to expand the anchor portion; i) deflating the distal balloon or other non-balloon expansive mechanism; j) removing the balloon catheter or other insertion device from the patient. Alternatively, the insertion sheath may be passed into the patient first and the balloon catheter and valve may be passed through the already inserted insertion sheath.

In an exemplary embodiment, the distal balloon of the balloon catheter may be deflated before or after deflation of the proximal balloon.

The guide wire may be placed in step (a) using any guide wire insertion method. For example, the guide wire may be placed using the techniques of transseptal catheterization, which involves floating a balloon catheter in the direction of blood flow through the left atrium (LA), left ventricle (LV), and into the aorta, which is then retrogradely snared. In a version of the conventional technique, the insertion sheath is advanced into the left atrium (LA) using its own dilator. The dilator is pulled out and the balloon catheter is then advanced through the sheath and exteriorized in the left atrium (LA). Once in the left atrium (LA), a balloon on the balloon catheter is inflated and floated out of the left ventricle (LV) through the aortic valve into the descending aorta, across the aortic arch and into the descending aorta. The wire is then be passed through the floating balloon catheter and exteriorized in the descending aorta. Once the balloon catheter is exteriorized, a retrograde advanced snare device is advanced retrogradely through the femoral artery and snares the tip of the wire and exteriorizes the wire out through the femoral artery, thereby completing the loop through the heart from the femoral vein to the femoral artery. See, for example, Babic et al., Percutaneous Mitral Valvuloplasty: Retrograde, Transarterial Double-Balloon Technique Utilizing the Transseptal Approach, Catheterization and Cardiovascular Diagnosis, 14:229-237 (1988), herein incorporated by reference in its entirety. In another embodiment, the transseptal sheath is sufficiently large to enable passage of the guidewire and splittable/two-part sheath through it into the ascending aorta.

The anchor portion may be self-expandable in which case when a balloon catheter is used it need only have a single balloon for inflation of the valve portion of the valve device. Alternatively, the distal balloon may be used in conjunction with a self-expandable anchor portion, for example, to assure complete expansion of the anchor portion.

The insertion sheath may include proximal and distal portions that are releasably connectable to each other.

The proximal and distal portions may be releasably connected by a threaded connection and may be configured such that separation of the proximal and distal portions is accomplished by rotating the proximal and distal portions relative to each other about a longitudinal axis of the sheath.

The proximal and distal portions of the sheath may also be releasably connected by a magnetic connection. For example, at least one of the proximal and distal portions may include a magnet, e.g., an electromagnet, and the other of the proximal and distal portions may include a magnetically-attractable member, a permanent magnet, an electromagnet, etc.

The proximal and distal portions of the sheath may also be connected by a latch. The latch may be integral with or connected to one of the proximal and distal portions and may fit in a recess in the other of the proximal and distal portions of the sheath so as to connect the proximal and distal portions of the sheath together. The latch may be triggered manually by pulling on a line, running a length of the sheath, which pivots the latch out of its mating recess. The latch may also be controlled by a motor or servo connected to the sheath. A line connected along a length of the sheath may communicate a control signal to the motor or servo from a controller so as to trigger the opening and closing of the latch.

The insertion sheath may also be configured to split, for example, circumferentially, into proximal and distal portions at a predetermined location on the insertion sheath upon application of a pulling force on opposite ends of the sheath or upon twisting of the proximal and distal portions relative to each other. The sheath may be weakened at the predetermined location relative to other locations along the sheath so as to facilitate separation of the sheath into two parts and to assure that separation of the sheath occurs at the predetermined location. For example, the sheath may have a reduced wall thickness or may be partially cut at the predetermined location so as to facilitate separation of the sheath into two parts and to assure that the separation occurs at the predetermined location.

An exemplary valve system of the present invention includes a valve and a sheath sized for insertion of the valve. The valve may include an anchor portion, a valve portion spaced apart from the anchor portion, at least one connector connecting the anchor portion and the valve portion and adapted to support the valve in the bodily vessel. The valve may be expandable and configured to be delivered into the bodily vessel through the insertion sheath in a low profile. The insertion sheath may be configured to separate or split into two parts so as to deploy the valve.

Exemplary embodiments of the present invention are described in more detail below with reference to the appended Figures.

### BRIEF DESCRIPTION OF THE DRAWINGS

FIG. 1 is a side view of a valve according to an exemplary embodiment of the present invention.

FIG. 2A is a perspective view of the valve portion of the valve of FIG. 1 in a closed state shown without an optional cloth covering and the connectors and with a portion of the valve wall removed.

FIG. 2B is a perspective view of the valve portion of the valve of FIG. 2A in an open state.

FIG. 2C is a perspective view of the valve portion of the valve of FIG. 1 in an open state shown without an optional cloth covering and the connectors.

FIG. 2D is a perspective view of the valve portion of the valve of FIG. 2C in a closed state.

FIG. 3 is a cross-sectional view of the valve taken along line 3-3 in FIG. 1 showing the cross-sectional shape of an exemplary embodiment of the connectors.

FIG. 4 is a cross-sectional view of the valve taken along line 4-4 in FIG. 1 showing an exemplary connection between a connector and the valve portion.

FIG. 5A is a side view of a valve according to an exemplary embodiment of the present invention.

FIG. 5B is a side view of a valve according to an exemplary embodiment of the present invention.

FIG. 6 illustrates a valve mounted on a balloon catheter inserted into the aorta and positioned for deployment of the valve.

FIG. 6A is a transverse cross-sectional view of the balloon catheter taken along line 6A-6A in FIG. 6.

FIG. 7 illustrates a valve mounted on a balloon catheter inserted into the aorta with one balloon inflated and expanding the stent.

FIG. 8 illustrates a valve mounted on a balloon catheter inserted into the aorta with both balloons inflated expanding both the stent and the valve portion.

FIG. 9 illustrates a valve fully expanded and secured in the aorta.

FIG. 10 illustrates a self-expandable valve mounted in a proximal end of a catheter inserted into the aorta and positioned for deployment of the valve.

FIG. 10A is a transverse cross-sectional view of the catheter taken along line 10A-10A in FIG. 10.

FIG. 11 illustrates the catheter of FIG. 10 with the sheath partially retracted and the valve partially expanded in the aorta.

FIG. 12a is a cross-sectional view of the heart and vasculature and a side view of an insertion sheath inserted therein over a guidewire.

FIG. 12b illustrates the insertion sheath of FIG. 12a with the proximal portion partially retracted and with a proximal balloon of a balloon catheter extending through the sheath inflated and expanding the valve portion of the valve.

FIG. 12c illustrates the balloon catheter of FIG. 12b with the entire insertion sheath removed and the distal balloon inflated expanding the anchor portion of the valve.

FIG. 12d is a side view of the valve implanted into the aorta.

FIG. 13 is a longitudinal cross-sectional view of a threaded connection connecting proximal and distal portions of the insertion sheath.

FIG. 14 is a longitudinal cross-sectional view of another threaded connection of the insertion sheath.

FIG. 15 is a perspective view of the insertion sheath including a magnetic connector system connecting proximal and distal portions of the insertion sheath.

FIG. 16 is a longitudinal cross-sectional view of a latch connection connecting proximal and distal portions of the insertion sheath.

FIG. 17 is a longitudinal cross-sectional view of another exemplary latch connection connecting proximal and distal portions of the insertion sheath.

### DETAILED DESCRIPTION

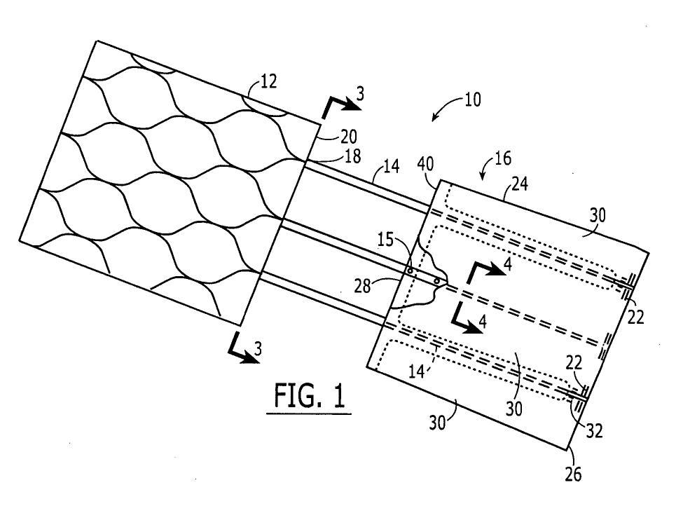

FIG. 1 illustrates a valve 10 of an exemplary embodiment of the present invention including an anchor portion 12, connectors 14 and a valve portion 16 spaced a distance away from anchor portion 12. Connectors 14 are connected on a distal end 18 to a proximal end 20 of anchor portion 12. Connectors 14 may extend at least partially along the length of the anchor portion 12. Connectors 14 may be connected to anchor portion 12, for example, by welding, suturing, gluing, clipping, rivets, etc. Connectors 14 may also be integral with anchor portion 12.

Connectors 14 extend along the commissural lines of the valve portion 16 a sufficient length so as to assure a strong connection with the valve portion 16. The connectors 14 may also be connected to the valve portion 16 at different points along its circumference. The valve portion 16 may be covered by a cloth 24 made from, for example, DACRON®, but also may be used without such covering. The portion of the connectors 14 connected to the valve portion 16 may lie between the cloth 24 and the valve portion 16, as shown, or may be connected to an inner or outer surface of the anchor portion 16. The connectors 14 may include ribs, such as T-shaped ribs 22, shown in dashed, to provide additional support to a proximal end 26 of the valve portion 16 and also to further secure connection of the connectors 14 to the valve portion 16. The valve portion 16 may be tapered towards the anchor portion 12. Further, the connectors 14 may include bores 15 for passage of sutures to connect to the valve portion 16. The connectors 14 may be manufactured by injection molding, machining, using nano-synthesized metals, etc.

The valve portion 16 is supported solely via its connection to the connectors 16 and is, in effect, suspended by the anchor portion 12. Valve portion 16 does not have an additional stent disposed within or over tubular portion 28, which, as indicated above, may adversely affect the performance of the valve 10. That is, tubular portion 28 and cloth 24 are stentless. Alternatively, the valve portion 16 may include a stent to maintain the valve portion 16 in the expanded position.

Valve portion 16 may be made from biological materials, such as (i) small intestine sub-mucosa (SIS), (b) large tubular vascular structure, e.g., IVC, superior vena cava (SVC), aorta, etc., (c) pericardial tissue, (d) fascia lata, (e) nano-synthesized material, such as Nitinol, (f) or other biocompatible materials such as urethane, polyurethane, polyethylene terephthalate (PET), polytetrafluoroethylene (PTFE), expanded PTFE, silk, Rayon, DACRON®, etc. The valve portion 16 may also be made from a suitable plastic, for example, such as Elast-Eon™, a metal, metal alloy, etc.

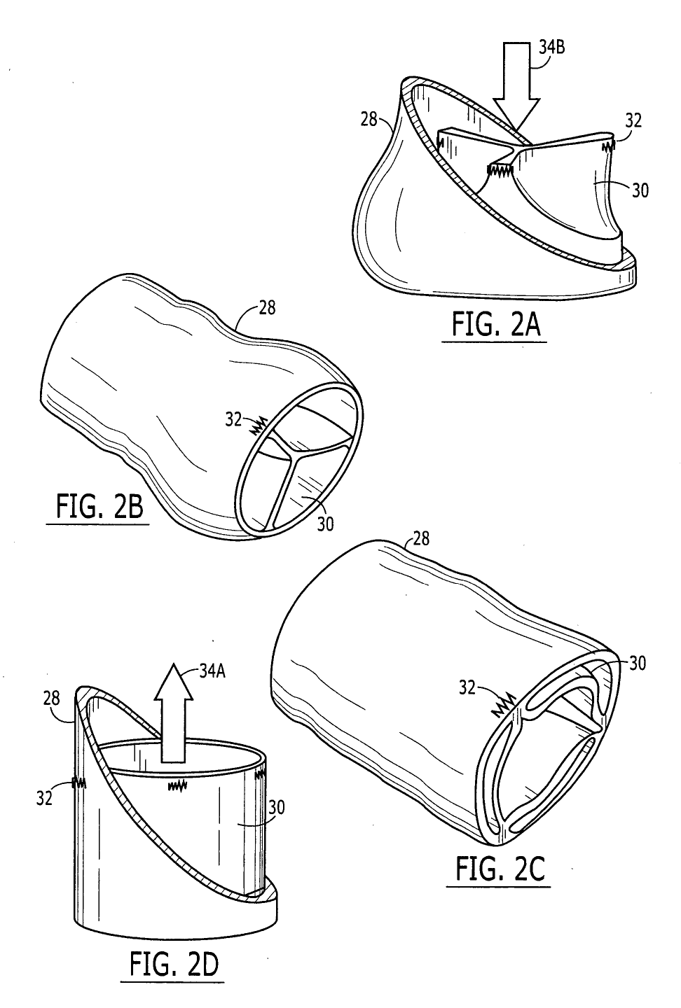

As illustrated in FIGS. 2A-2D, the valve portion 16, shown without optional cloth 24, includes a tubular portion 28 and flaps 30. FIGS. 2A-2D illustrate the valve portion in open and closed states. A portion of the tubular portion 28 is removed in FIGS. 2A and 2D so as to expose the flaps 30. The valve portion 16 is shown having a tricuspid configuration but may also have a bicuspid configuration. Further, flaps 30 are shown having a rectangular shape but may have any suitable size and configuration, e.g., triangular, etc. The specific number of flaps and the size and configuration chosen for the flaps 30 will depend on the size, configuration, and/or nature of the vessel in which the valve 10 will be implanted. Flaps 30 move from an opened position in which they extend substantially parallel with the tubular portion 28 and, thus allow blood flow along arrow 34A, as shown in FIGS. 2C and 2D, and a closed position, as shown in FIGS. 2A and 2B, in which the flaps 30 contact each other and, thus, prevent flow in one direction along arrow 34B across the valve portion 16. Valve portion 16 may be formed, for example, by invaginating a tubular structure, suturing the ends together at one or more suture points 32, and incising an inner wall of the invaginated tubular structure in at least two locations so as to form leaflets or flaps 30, which permit unidirectional blood flow.

Each of the flaps 30 may be constructed so as to form a pouch cavity, which fills with blood when the valve 10 is closed. This construction minimizes paravalvular leaks by a mechanism similar to a hyrdrofoil.

Anchor portion 12 may be a collapsible and radially re-expandable support, such as a stent, made from, for example, Nitinol, stainless steel, for example, such as NP-35N alloy, etc. Anchor portion 12 may include markers, such as heavy metal markers, to facilitate placement within the body. Anchor portion 12 may include, for example, a gold, platinum, iridium tantalum or similar metal, etc., as a marker. The diameter of the anchor portion 12 may be, for example, between 4 mm and 50 mm. Anchor portion 12 may be cylindrical or may have a truncated conical form tapering towards the valve portion 16. Anchor portion 12 may include structural features, such as barbs, that help maintain its position in the vessel following implantation.

Anchor portion 12 is illustrated in FIG. 1 as having a sinusoid configuration but may have any type of cell design including, for example, zig-zag elements, ring members, braided strands, helically wound strands, consecutively attached ring members, tube members, a frame cut from solid tubes, etc. Further, the anchor portion 12 may be configured such that it is larger in diameter than the inner diameter of the vessel in which it will be implanted so as to facilitate maintenance of the valve 10 in the vessel.

Additional examples of suitable anchor portions for use with valve 10 include those described in U.S. Pat. No. 6,508,833 to Pavcnik et al., entitled “Multiple-sided Intraluminal Medical Device,” U.S. Pat. No. 6,464,720 to Boatman et al., entitled “Radially Expandable Stent,” U.S. Pat. No. 6,231,598 to Berry et al., entitled “Radially Expandable Stent,” U.S. Pat. No. 6,299,635 to Frantzen, entitled “Radially Expandable Non-Axially Contracting Surgical Stent,” U.S. Pat. No. 4,580,568 to Gianturco, entitled “Percutaneous Endovascular Stent and Method for Insertion Thereof,” and U.S. Patent Application Publication No. 2001/0039450 to Pavcnik et al., entitled “Implantable Vascular Device,” each of which is expressly incorporated herein in its entirety by reference thereto.

A resorbable material may also be used for the anchor portion 12. A number of resorbable materials are believed to be conventional, and any suitable resorbable material may be used. Examples of suitable types of resorbable materials include resorbable homopolymers, copolymers, blends of resorbable polymers, etc. Specific examples of suitable resorbable materials include poly-alpha hydroxy acids, such as polylactic acid, polylactide, polyglycolic acid (PGA), and polyglycolide, trimethylene carbonate, polycaprolactone, poly-beta hydroxy acids, such as polyhydroxybutyrate or polyhydroxyvalerate, and other polymers such as polyphosphazines, polyorganophosphazines, polyanhydrides, polyesteramides, polyorthoesters, polyethylene oxide, polyester-ethers (e.g., polydioxanone), polyamino acids (e.g., poly-L-glutamic acid or poly-L-lysine), etc. There are also a number of naturally derived resorbably polymers that may be suitable, including modified polysaccharides, such as cellulose, chitin, and dextran, and modified proteins, such as fibrin and casein, etc.

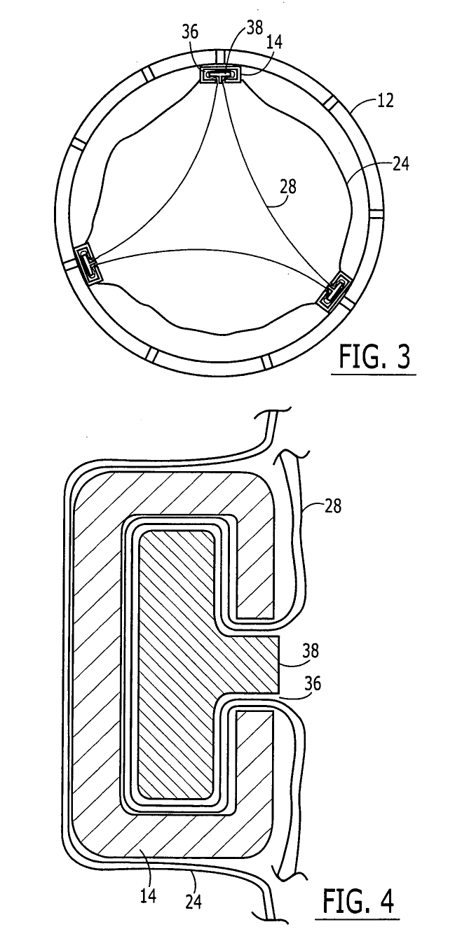

FIG. 3 is a cross-sectional view of valve 10 taken along line 3-3 in FIG. 1. As illustrated in FIG. 3, connectors 14 have a roughly C-shaped cross section with a slot 36.

The connectors 14 may be connected to the valve portion 16, for example, by suturing, stapling, riveting and chemical adhesion, etc. Connectors 14 may also be connected to the valve portion 16 mechanically, as illustrated in FIG. 4. FIG. 4 is a cross-sectional view taken along line 4-4 in FIG. 1. As illustrated in FIG. 4, a T-shaped member 38 is slid into slot 36 along with tubular portion 28 thereby securing connector 14 to valve portion 16 via tubular portion 28. T-shaped member 38 may be sized and shaped so as to assure a snug fit within slot 36. As indicated above, connector 14 may be connected to valve portion 16 using suturing, stapling, riveting and chemical adhesion, in which case, the cross-section of the connector 14 may not need to have slot 36 and may have any other shape.

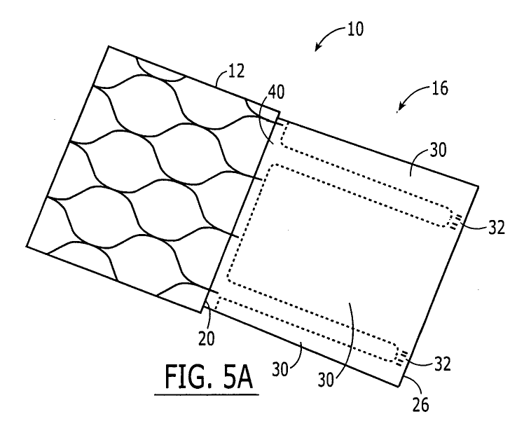

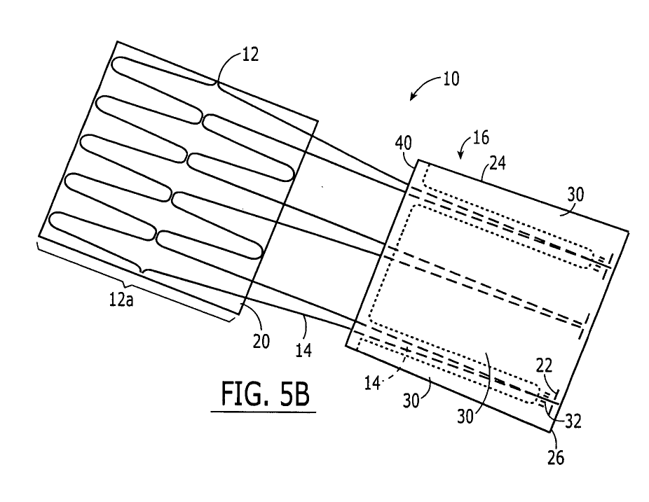

FIG. 5A illustrates a valve similar to that illustrated in FIG. 1 except that the valve portion 16 is directly connected on its distal end 40 to the proximal end 20 of the anchor portion 12 via, for example, sutures, staples, rivets, chemical adhesion, etc. Valve portion 16 is supported solely via its connection on its distal end 40 of the anchor portion 12 and is, in effect suspended by the anchor portion 12. As in the example embodiment illustrated in FIG. 1, valve portion 16 does not have an additional stent disposed within or over tubular portion 28, which, as indicated above, may adversely affect the performance of the valve 10. That is, tubular portion 28 is stentless. Alternatively, and as indicated above, the tubular portion 28 may include a stent to maintain its expanded position.

FIG. 5B illustrates an exemplary embodiment similar to that illustrated in FIG. 1 except that the anchor portion 12 has a horizontal sinusoidal configuration and the connectors 14 are integral with the stent. The anchor portion 12 has a main body portion 12a and connectors 14 that are integral with the stent and extend beyond a proximal end 20 of the body portion 12a. The valve portion 16 is connected to the connectors 14 such that a gap exists between the body portion 12a and the valve portion 16. The longer the gap, and the fewer the number of connectors 14, the less the expansion of the body portion 12a may affect the functioning of the valve portion 16. The above applies to the exemplary embodiments illustrated in FIGS. 1 and 5 as well. Further, with respect to the example embodiment illustrated in FIG. 5B, the larger the number of sinusoids in the main body portion 12a, the less the expansion of the body portion 12a may affect the functioning of the valve portion 16.

When the valve is used as a cardiac valve prosthesis in the aorta or main pulmonary artery, it is possible to mount the valve proximal to the native valve, within the native cardiac valve (with or without stenting of the native valve) or distal to the native valve, e.g., in the ascending aorta, descending aorta or distal the main pulmonary artery. The valve may be used in place of the tricuspid valve, mitral valve and in artificial or biological conduits that may connect different chamber in the cardiovascular system, e.g., right ventricle to pulmonary artery conduits, intracardiac or extracardiac Fontan connections, left ventricle (LV) to ascending aorta, etc.

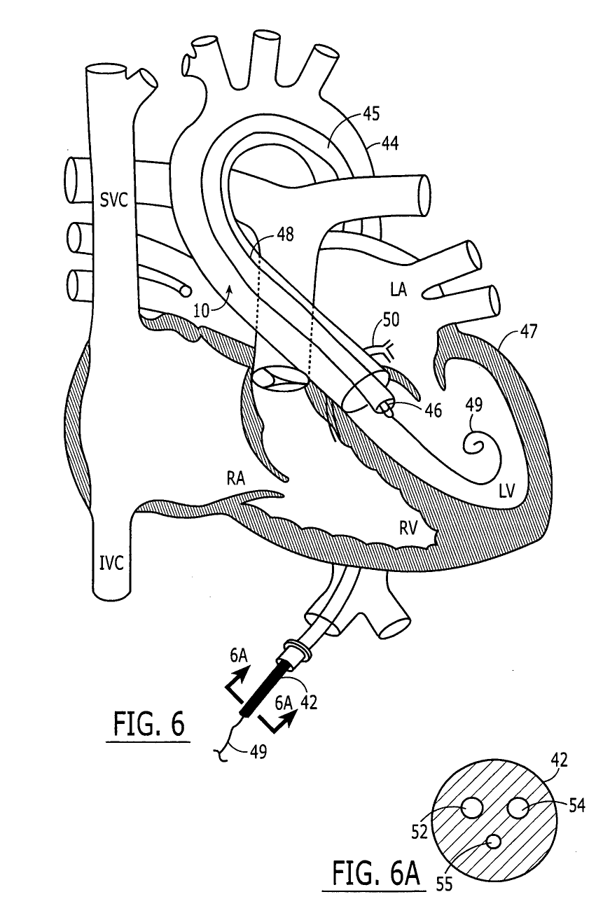

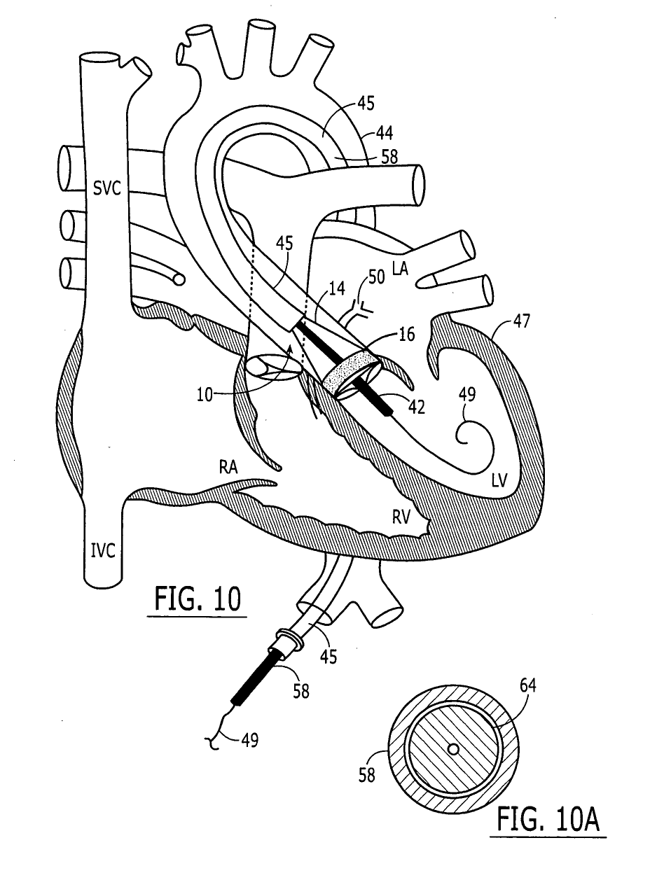

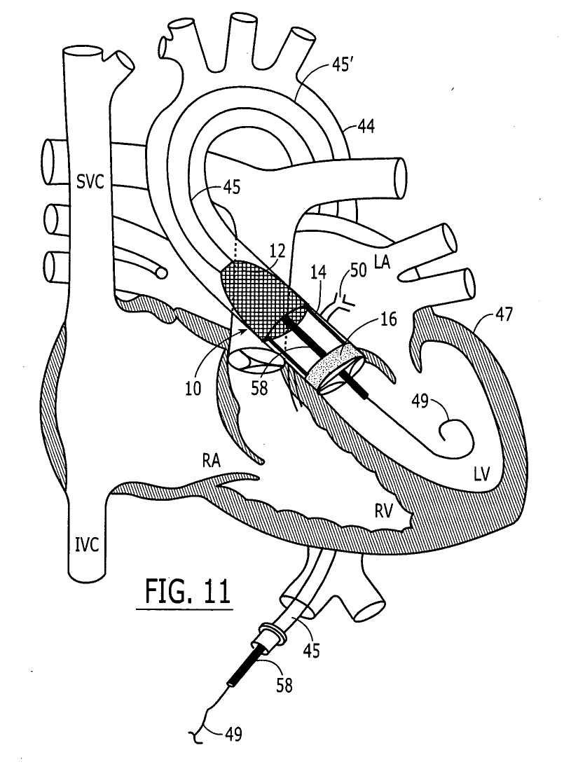

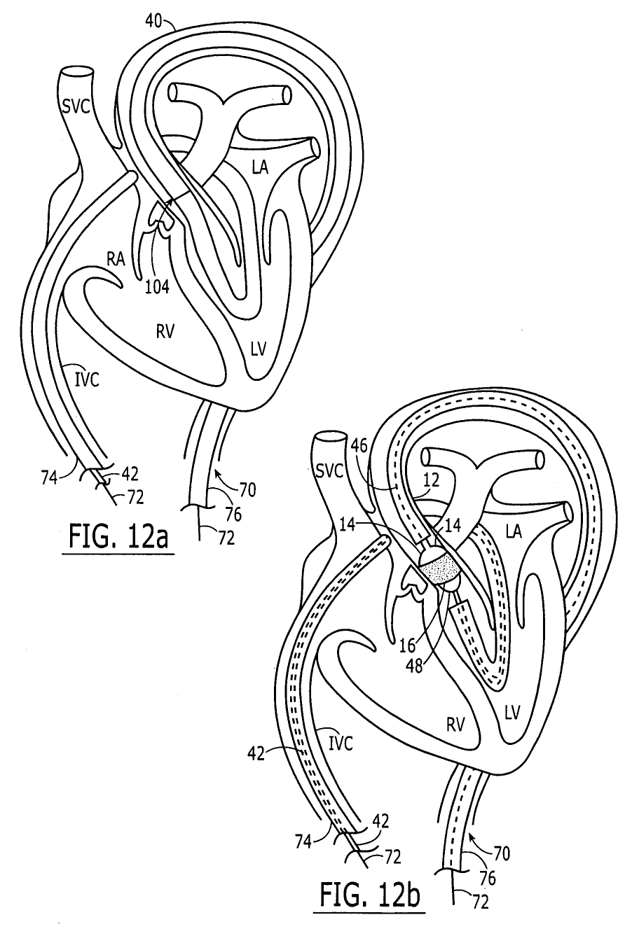

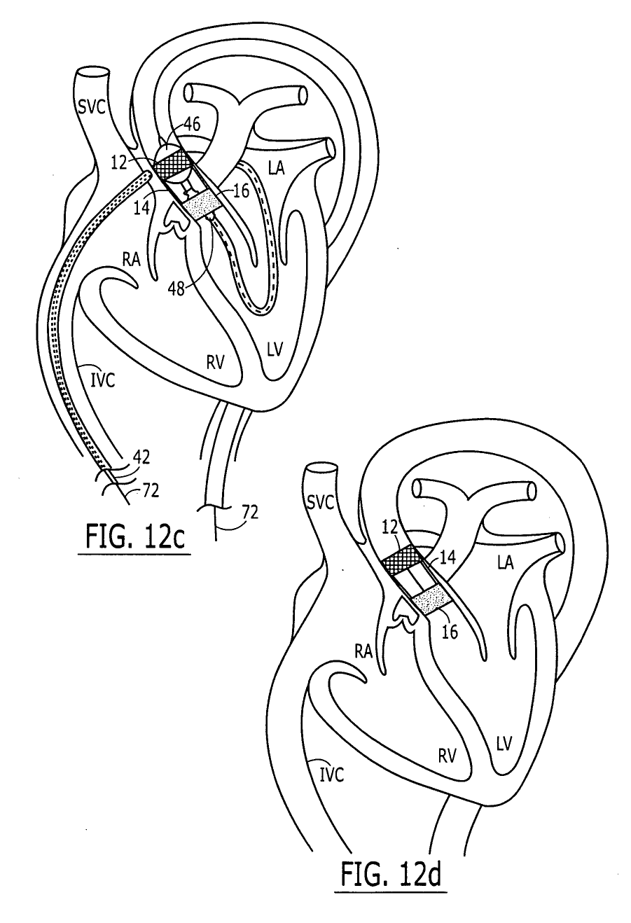

The valve 10 may be surgically implanted in a bodily vessel or inserted percutaneously via a catheterization procedure, which may be significantly less invasive than open surgery. FIGS. 6 to 9 illustrate insertion steps for insertion of an expandable valve using a balloon catheter. FIGS. 10 and 11 illustrate insertion steps for insertion of a self-expanding valve using a catheter having a retractable sheath. FIGS. 12a-12d illustrate insertion steps for insertion of an expandable valve using a sheath splittable or separable into two separate sheaths.

In the methods illustrated in FIG. 6 to 11, the valve 10 is illustrated implanted using a retrograde approach, e.g., approaching the aortic valve from the descending aorta, but may also be delivered using an antegrade approach, e.g., approaching the aortic valve from the left ventricle (LV) after performing, for example, a transseptal puncture.

FIG. 6 illustrates a balloon catheter 42 inserted into the aorta 44 through a sheath 45 and positioned such that a first balloon 46 is adjacent the heart 47 at or near the anatomical location of the native aortic valve (which may be removed in the case of calcific aortic stenosis, regurgitation) and an independently inflatable second balloon 48 is just downstream of the ostiums of the coronary arteries 50. A guide tool, such as a guide wire 49, may be used to guide the balloon catheter 42 to the position illustrated in FIG. 6. Further, X-ray supervision, injection of X-ray traceable liquids, intravascular or intracardiac ultrasound, ultrasonic measuring, etc., may also be used to assist in positioning the valve 10. The heart 47, aorta 44 and coronary arteries 50 are illustrated in longitudinal cross-section. As illustrated in FIG. 6, which illustrates the transverse cross-section of balloon catheter 42 taken along line 6A-6A in FIG. 6, balloon catheter 42 may have a first lumen 52 in communication with first balloon 46 for inflation of the first balloon 46, a second lumen 54 for inflation of the second balloon 56, and a guide wire lumen 55. Valve portion 16 is radially compressed and/or folded over the first balloon 46, and anchor portion 12 is radially compressed and/or folded over the second balloon 56. Thus, anchor portion 12 and valve portion 16 are delivered into the aorta 44 in a low profile configuration.

Valve 10 may be folded and radially compressed using, for example, a crimping device including a plurality of adjustable plates resembling a typical single lens reflex (SLR) camera variable restrictor. Each plate moves along a line passing off an opening in the center, and all plates are equidistant from the center opening. The plates may be adapted to move simultaneously by a lever and transmission.

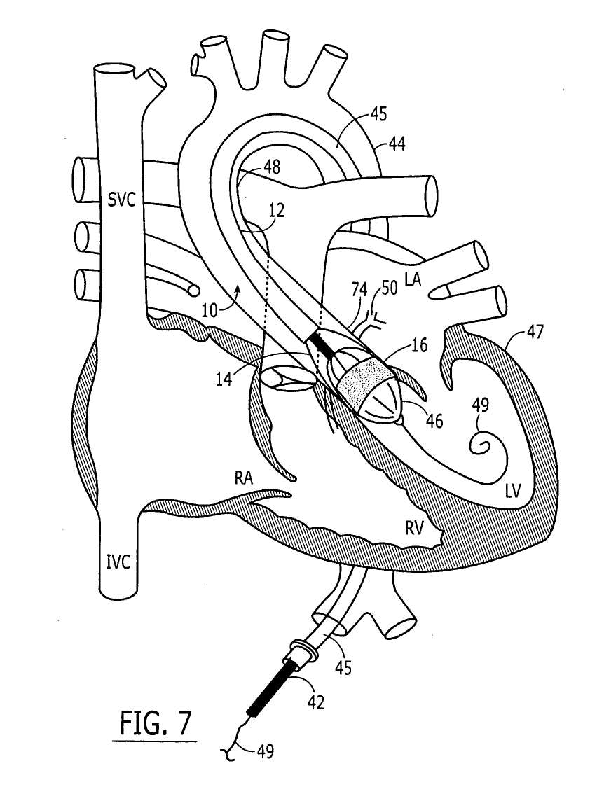

As illustrated in FIG. 7, sheath 45 is partially withdrawn to a position distal the first balloon 46 and the first balloon 46 is inflated so as to expand valve portion 16 to its larger profile configuration, such that its profile matches that of the aorta 44.

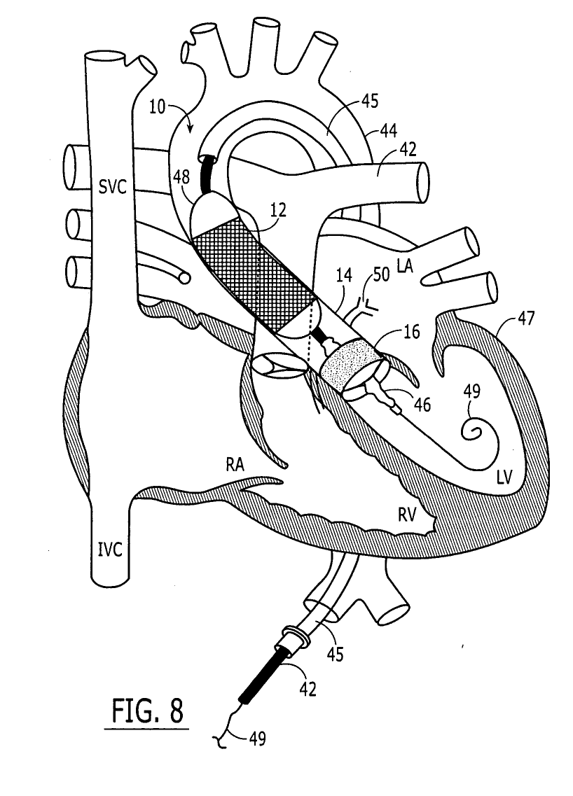

As illustrated in FIG. 8, sheath 45 is further withdrawn to a position distal the second balloon 48 and the second balloon 48 is inflated so as to expand the anchor portion 16 such that its profile is slightly larger than that of the aorta 44. Anchor portion 12 anchors the valve 10 in position. The first balloon 46 may be deflated before inflating the second balloon 48 so as to minimize interruption of blood flow.

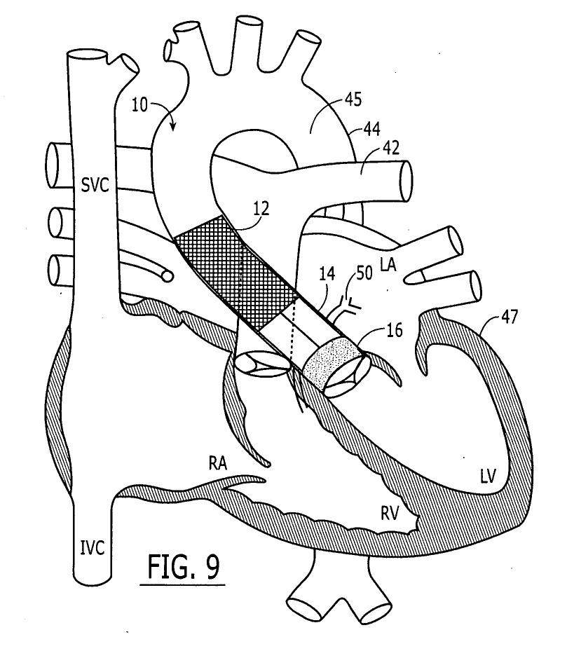

FIG. 9 illustrates the state after the balloon catheter 42 is removed from the aorta 44 and the valve 10 is fully expanded and secured in place.

The placement of the valve 10 in the aorta 44 may need to be precise in order to avoid blocking the opening to the coronary arteries 50, which branch off the aorta 44. Separation of the anchor portion 12 and the valve portion 16 may allow for the use of a shorter valve portion and may facilitate placement of the valve portion 16 in the aorta 44 without blocking the coronary arteries 50 by the valve portion 16 or the anchor portion 12. In traditional valves having stents disposed within or over the valve, the valves may need to be long enough to accommodate a stent of sufficient length to assure fixation and support of the valve. In accordance with an example embodiment of the present invention, separation of the valve and the stent may allow for the use of a shorter valve and, thus, may provide a surgeon more leeway in placement of the valve because the connectors 14 may be placed adjacent the opening of the coronary arteries 50 without presenting any danger of blockage.

Alternatively, sheath 45 may be initially withdrawn distal to both the first balloon 46 and second balloon 48 and the balloons 46, 48 may be inflated simultaneously or the second balloon 56 may be inflated before first balloon 46. In an exemplary embodiment of the present insertion method, balloon catheter 42 may have only a single balloon. Valve portion 16 may not need to be expanded by a balloon because blood flow in the aorta 44 may cause valve portion 16 to fully expand. Anchor portion 12 may be self-expandable and, therefore, may also not need to be expanded by a balloon.

FIG. 10 illustrates a catheter 58 having a self-expandable valve 10 packed within a proximal portion of a retractable sheath 45. Retraction of the sheath 45 relative to plunger 64 exposes valve 10 within the aorta 44. Valve portion 16 may be self-expandable and, thus, upon retraction of sheath 45, may spring open to a larger profile matching that of the aorta 44. FIG. 11 illustrates the state after the sheath 45 is partially withdrawn from the anchor portion 16. The anchor portion 16 may be self-expandable and, thus, may expand, as illustrated in FIG. 11, as the sheath 45 is being withdrawn. Balloon 66 on a proximal end of the balloon catheter 58 may be inflated after complete withdrawal of sheath 45 from over anchor portion 12 so as to ensure complete expansion of the anchor portion 12 to a profile slightly larger than the profile of the aorta 44. Upon complete expansion of the valve 10 the balloon catheter 58, sheath 45 and guide wire 66 are removed from the patient leaving the valve anchored within the aorta 44.

The valve 10 may also be implanted using a sheath 70 that is separable or splittable into two parts. FIGS. 12a to 12d illustrate exemplary insertion steps for the valve 10 using separable or splittable sheath 70.

FIG. 12a shows the sheath 70 inserted into a patient over the guide wire 72. The patient's heart and vasculature are shown in cross-section. The guide wire may be placed using any guide wire insertion method. For example, the guide wire may be placed using the techniques of transseptal catheterization, which involves floating a balloon catheter in the direction of blood flow through the left atrium (LA), left ventricle (LV), and into the aorta 44, which is then retrogradely snared. In a version of the conventional technique, the insertion sheath is advanced into the left atrium (LA) using its own dilator. The dilator is pulled out and the balloon catheter is then advanced through the sheath and exteriorized in the left atrium (LA). Once in the left atrium (LA), a balloon on the balloon catheter is inflated and floated out of the left ventricle (LV) through the aortic valve into the descending aorta, across the aortic arch and into the descending aorta. The wire is then passed through the floating balloon catheter and exteriorized in the descending aorta. Once the balloon catheter is exteriorized, a retrograde advanced snare device is advanced retrogradely through the femoral artery and snares the tip of the wire and exteriorizes the wire out through the femoral artery, thereby completing the loop through the heart from the femoral vein to the femoral artery. See, for example, Babic et al., Percutaneous Mitral Valvuloplasty: Retrograde, Transarterial Double-Balloon Technique Utilizing the Transseptal Approach, Catheterization and Cardiovascular Diagnosis, 14:229-237 (1988), which is expressly herein incorporated by reference in its entirety. The transseptal sheath is sufficiently large to enable passage of the guidewire 72 and removal of the distal portion of the separable/splittable sheath 70 through it from the ascending aorta 44.

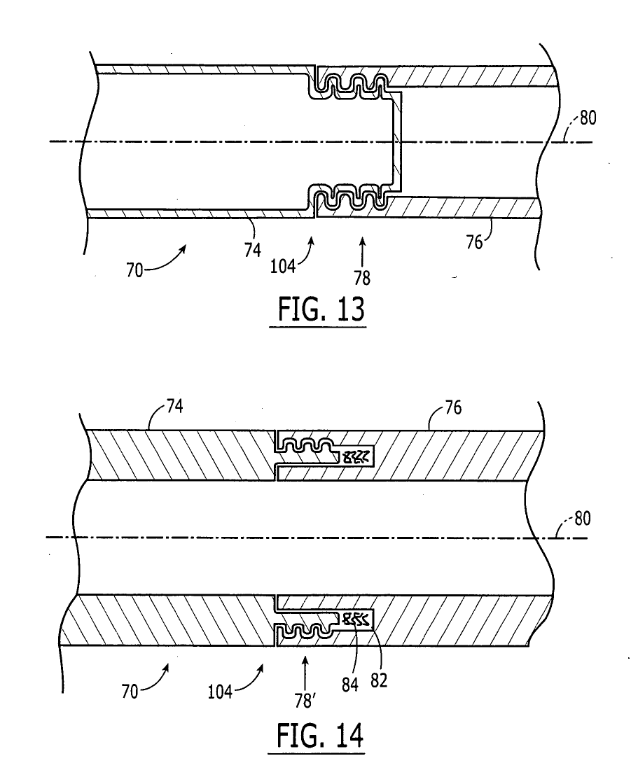

The sheath 70 may be separable into a distal portion 76 and a proximal portion 74. A distal end of the proximal portion 74 and a proximal end of the distal portion 76 may be releasably connectable. For example, the proximal and distal portions 74, 76 may be connected via a threaded connection 78, as illustrated in FIG. 13. The sheath 70 may be separated into the proximal and distal portions 74, 76 by rotating these portions in opposite directions about a longitudinal axis 80 of the sheath 70.

FIG. 14 illustrates a sheath 70 with a threaded connection 78′. The sheath 70 may include a pocket 82 for delivery of a medical device, e.g., valve 10, or drug 84 into the body of the patient. Pocket 82 is opened upon disconnection of the distal portion 74 and the proximal portion 76 of the sheath 70. Pocket 82 may be internally threaded to receive an end of the proximal portion 74, which may also be threaded.

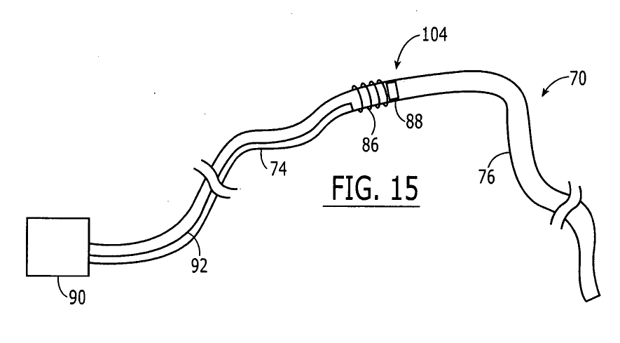

The proximal and distal portions 74, 76 may be magnetically connected, as illustrated in FIG. 15. A coil 86 may be connected, for example, to an end of the proximal portion 74 and a permanent magnet 88 may be connected, for example, to an end of the distal portion 76. To secure the ends of the proximal and distal portions 74, 76 together a current is passed through the coil 86 to generate a magnetic field which is attracted to the magnetic field produced by the permanent magnet 88. A controller 90, may be used to control the current supplied to coil 86 via line 92. The permanent magnet 88 may be replaced by a second coil and controller, such that both portions of the sheath 70 include an electro-magnet. The coil 86 and line 92 are illustrated as connected to an outer surface of the sheath 70 but they may also be connected to an inner surface of the sheath 70, embedded within the sheath 70, or extend through a lumen in a wall of the sheath 70.

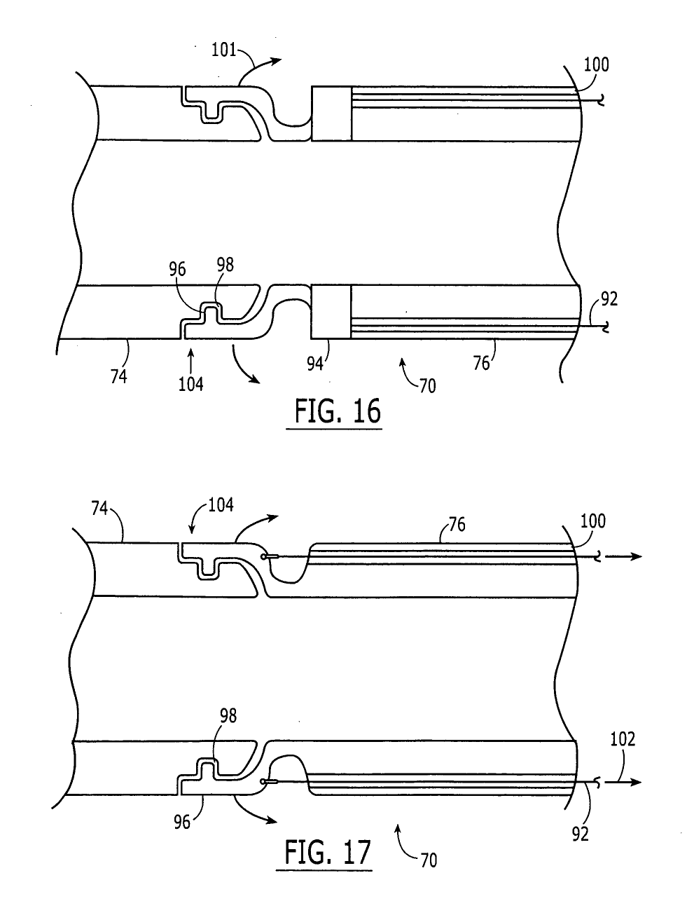

As illustrated in FIG. 16, line 92 may be connected to a motor or servo 94 used to control a latch 96. Latch 96 may move in the direction of arrow 101 between a connected position illustrated in FIG. 16, in which the latch 96 sits in a slot 98, and an unconnected position in which latch 96 is pivoted by motor or servo 94 out of slot 98. Controller 90 may be used to power the motor or servo 94 and, thus, open and close latch 96.

Line 92 may also be used to manually pivot the latch 96 between a locked and unlocked position. As illustrated in FIG. 17, line 92 may be slidingly disposed within lumen 100 and may connect at one end to latch 96. Pulling line 92 in a direction of arrow 102 may pivot latch 96 and disconnect the proximal and distal portions 74, 76 of sheath 70.

The sheath 70 may be positioned such that contact point 104, i.e., the location where the connecting ends of the proximal and distal portions 74, 76 come together, is located in the patient at the desired deployment site for the valve 10, for example, near the anatomical location of the native aortic valve. An insertion device, such as balloon catheter 42, as illustrated in FIGS. 12b and 12c, may be advanced over the guide wire 72 through the sheath 70 such that distal balloon 46 is located on one side of the contact point 104 and proximal balloon 48 is positioned on an opposite side of the contact point 104. The valve portion 16 of the valve 10 may be disposed over the proximal balloon 48 and the anchor portion 12 may be disposed over the distal balloon 46. As an alternative to placement of the sheath 70 first and then advancing the balloon catheter 42 into position within the sheath 70, the balloon catheter 42 may be disposed within the sheath 70 and advanced into position over guidewire 72 together with the sheath 70.

The proximal portion 74 of the sheath 70 may be shifted away from the distal portion 76 towards a femoral artery cannulation site, thus exposing the valve portion 16 of the valve 10, as illustrated in FIG. 12b. The proximal balloon 48 may be inflated so as to expand the valve portion 16 in the aorta 44. The connectors 14 may be of sufficient length to allow the valve portion 16 to fully expand while the anchor portion 12 remains in a low profile state within sheath 70.

The proximal balloon 48 may be deflated and the distal portion 76 of the sheath 70 may be withdrawn from the patient, for example, through the venous system, thus exposing the anchor portion 12 of the valve 10. The distal balloon 46 may then inflated so as to expand the anchor portion 12, as illustrated in FIG. 12c. The distal balloon 46 may be deflated and then removed from the patient, for example, through the venous system. Anchor portion 12 may also be self-expandable, in which case the distal balloon 46 may not be necessary but may still be used to assure complete expansion of the anchor portion 12. Thus, if a self-expandable anchor portion 12 is used, the balloon catheter 42 may have a single balloon. FIG. 12d illustrates the implanted valve 10 after the sheath 70, balloon catheter 42 and guide wire 72 have been completely removed from the patient.

Rather than entirely removing the proximal portion 74 of the sheath 70 so as to expose the valve portion 16, the proximal portion 74 may be partially removed (enough to completely expose the valve portion 16) and then may be removed together with the balloon catheter 42 after the valve 10 is fully implanted.

Although explained in connection with cardiac heart valves implanted in the aortic position, the valve 10 may be implanted using similar implantation techniques in other non-cardiac vessels or in other channels in the body, for example, in the veins, esophagus, stomach, ureter, bladder, urethra, biliary passes, lymphatic system, intestines, in CNS shunts and in the Fallopian tubes or other portions of the reproductive system, etc. The valve prosthesis may be used to replace a natural valve or to establish a new valve function in one of the channels in the body that does not naturally include a valve. The valve may be arranged to ensure that fluids, such as blood, flows in only one direction through the valve. In persons having varicose veins, the blood flows in the wrong direction. A valve hereof may, for example, be placed in the varicose vein to prevent flow of blood in the wrong direction.

The foregoing description and example embodiments have been set forth for illustrative purposes only and are not intended to be limiting. Each of the disclosed aspects and example embodiments may be considered individually or in combination with other aspects, embodiments, and variations. Modifications of the described example embodiments may be made without departing from the spirit and scope hereof.

What is claimed is:

1. A valve for placement in a bodily vessel, comprising:
an anchor portion;
a valve portion spaced apart from the anchor portion; and
at least one connector connecting the anchor portion and the valve portion and adapted to support the valve in the bodily vessel.

2. The valve according to claim 1, wherein the valve portion and the anchor portion are configured to be delivered into the bodily vessel in a low profile and to be expanded to a larger profile, the anchor portion configured to anchor the valve to the bodily vessel.

3. The valve according to claim 1, wherein the anchor portion includes a stent.

4. The valve according to claim 1, wherein the anchor portion is self-expandable.

5. The valve according to claim 1, wherein the connector extends along substantially an entire length of the valve portion.

6. The valve according to claim 1, wherein the valve portion is substantially tubular and includes a plurality of flaps configured to allow fluid to pass therethrough in only one direction.

7. The valve according to claim 1, wherein the valve portion is made from at least one of (a) small intestine sub-mucosa, (b) large tubular vascular structure, (c) pericardial tissue, (d) fascia lata, (e) a nano-synthesized material, (f) silk, and (g) expanded polytetrafluoroethylene.

8. The valve according to claim 6, wherein the valve portion is made of an invaginated tube, an inner wall of the invaginated tube cut at at least two locations to form the flaps.

9. The valve according to claim 1, wherein the anchor portion is tapered toward the valve portion.

10. The valve according to claim 1, wherein the connector has a C-shaped cross-section.

11. The valve according to claim 10, further comprising a T-shaped retainer securing the valve portion to each connector.

12. The valve according to claim 11, wherein the T-shaped retainer is disposed within a slot in the connector, the valve portion arranged between each T-shaped retainer and connector.

13. The valve according to claim 1, wherein the valve portion is at least one of (a) glued, (b) sutured, (c) riveted, and (d) stapled to the connector.

14. The valve according to claim 1, wherein the connector is at least one of (a) integral with (b) chemically adhered, (c) sutured, (d) riveted and (e) stapled to the anchor portion.

15. The valve according to claim 1, wherein a portion of the connector in contact with the valve portion is ribbed.

16. The valve according to claim 1, wherein a portion of the connector in contact with the valve portion includes bores.

17. The valve according to claim 1, wherein the valve portion is stentless.

18. A valve for placement in a bodily vessel, comprising:
an anchor portion; and
a stentless valve portion connected end-to-end with the anchor portion and supported by at least one connection to the anchor portion.

19. A method, comprising:
deploying a valve in a bodily vessel, including inserting the valve into the bodily vessel and anchoring an anchor portion of the valve to the bodily vessel, the valve including at least one connector connecting the anchor portion to a valve portion of the valve, the valve portion spaced apart from the anchor portion.

20. The method according to claim 19, wherein the deploying includes:
arranging the anchor portion of the valve in the bodily vessel on one side of a branch leading into the bodily vessel; and
arranging the valve portion of the valve in the bodily vessel on a side of the branch opposite to the anchor portion, the at least one connector of the valve spanning the branch, the at least one connector arranged to permit fluid flow therethrough between the branch and the bodily vessel.

21. A method for implanting a valve into a bodily vessel, comprising:
(a) passing a balloon catheter having a first balloon on a proximal end into the bodily vessel, a valve portion of the valve mounted on the first balloon in a low-profile state;
(b) inflating the first balloon to expand the valve portion to a larger profile;
(c) deflating the first balloon;
(d) anchoring an anchor portion of the valve in the bodily vessel, the anchor portion spaced apart from and connected to the valve portion by at least one connector; and
(e) withdrawing the balloon catheter from the bodily vessel.

22. The method according to claim 21, wherein the anchor portion is self-expandable and is anchored in the bodily vessel in the anchoring step (d) by expansion from a low-profile state to a larger-profile state.

23. The method according to claim 21, wherein the balloon catheter includes a second balloon, the anchor portion disposed about the second balloon and anchored in the anchoring step (d) by inflating the second balloon.

24. The method according to claim 21, wherein the valve portion is supported in the bodily vessel by the connector.

25. The method according to claim 23, further comprising inflating the first balloon in the inflation step (b) before inflating the second balloon.

26. The method according to claim 21, wherein the valve portion is stentless.

27. The method according to claim 21, further comprising inserting a guidewire into the bodily vessel, the balloon catheter passing over the guidewire.

28. A method for implanting a valve into a bodily vessel, the valve including a self-expandable anchor portion, a valve portion spaced apart from the anchor portion and at least one connector connecting the anchor portion and the valve portion, comprising:
(a) passing a catheter into the bodily vessel, the catheter including a plunger and a sheath disposed over the plunger, the sheath extending beyond a proximal end of the plunger, the valve disposed within a proximal end portion of the sheath in a low-profile state;
(b) retracting the sheath relative to the plunger to withdraw the sheath from over the valve to expand the anchor portion in the bodily vessel to a larger profile; and
(c) withdrawing the catheter from the bodily vessel.

29. The method according to claim 28, wherein the valve portion is supported in the bodily vessel by the connector.

30. The method according to claim 28, wherein the valve portion is stentless.

31. The method according to claim 28, further comprising inserting a guidewire into the bodily vessel, the catheter passing over the guidewire.

32. A method for implanting a valve into a patient, comprising:
(a) inserting a sheath into the patient, the sheath including a proximal portion and a distal portion, a valve portion of the valve at least partially disposed within an outside surface of a first one of the proximal portion and the distal portion, an anchor portion of the valve at least partially disposed within an outside surface of a second one of the proximal portion and the distal portion; and
(b) separating the proximal portion and the distal portion to expose the valve.

33. The method according to claim 32, further comprising:
passing an insertion device into the patient, the valve device mounted to the insertion device, the separating step (b) including (i) shifting the proximal portion of the sheath away from the distal portion of the sheath to expose the valve portion of the valve and (ii) shifting the distal portion of the sheath away from the valve portion to expose the anchor portion of the valve; and
removing the insertion sheath and the insertion device from the patient.

34. The method according to claim 32, further comprising passing a guidewire into the patient, the sheath being inserted into the patient over the guidewire.

35. The method according to claim 34, wherein the guidewire is inserted through the femoral vein, inferior vena cava, right atrium, left atrium, left ventricle, ascending and descending aorta 44, abdominal aorta, and iliac artery, and is exteriorized through the femoral artery.

36. The method according to claim 32, wherein, prior to separating in the separating step (b), the sheath is positioned in the patient such that a distal end of the proximal portion and a proximal end of the distal portion of the sheath is adjacent to a deployment site for the valve.

37. The method according to claim 36, wherein the deployment site is in the aorta.

38. The method according to claim 32, wherein the separating in the separating step (b) includes splitting the sheath into the distal and proximal portions.

39. The method according to claim 32, further comprising, prior to the separating step (b), disconnecting a distal end of the proximal portion of the sheath and a proximal end of the distal portion of the sheath.

40. The method according to claim 33, wherein the insertion device includes a balloon catheter, and the anchor portion of the valve device is disposed over a distal balloon of the balloon catheter, the method further comprising:
inflating the proximal balloon, prior to shifting the distal portion of the sheath in the separating step (b); and
deflating the proximal balloon.

41. The method according to claim 34, wherein the proximal balloon is deflated prior to shifting of the distal portion of the sheath in the separating step (b).

42. The method according to claim 40, further comprising inflating a distal balloon of the balloon catheter to expand the anchor portion of the valve device after shifting of the distal portion of the sheath in the separating step (b).

43. The method according to claim 33, wherein shifting of the proximal portion of the sheath in the separating step (b) includes removing the proximal portion from the patient.

44. The method according to claim 33, wherein shifting of the distal portion of the insertion sheath in the separating step (b) includes removing the distal portion from the patient.

45. The method according to claim 32, wherein the anchor portion is self-expandable.

46. The method according to claim 32, wherein the anchor portion and valve portion are one of (i) connected end-to-end and (ii) spaced apart and connected by connector struts.

47. A valve system, comprising:
a valve including an anchor portion and a valve portion, the valve portion one of (i) spaced apart from the anchor portion and connected to the anchor portion by at least one connector adapted to support the valve in the bodily vessel, and (ii) connected end-to-end with the anchor portion; and
a sheath sized for insertion of the valve into a bodily vessel, the valve positionable within the sheath, the valve expandable and adapted to be delivered into the bodily vessel one of (i) through and (ii) in the sheath in a low-profile state, the sheath including a proximal portion and a distal portion that are separable to expose the valve.

48. The valve system according to claim 47, wherein the sheath includes proximal and distal portions that are releasably connected to each other.

49. The valve system according to claim 48, wherein the proximal and distal portions are releasably connected by a threaded connection.

50. The valve system according to claim 48, wherein the proximal and distal portions are releasably connected by a magnetic connection.

51. The valve system according to claim 50, wherein an end of a first one of the proximal portion and the distal portion includes a first coil configured to generate a first magnetic field, and an end of a second one of the proximal portion and the distal portion includes one of (i) a second coil configured to generate a second magnetic field and (ii) a permanent magnet.

52. The valve system according to claim 48, wherein the proximal and distal portions are releasably connected by a latch.

53. The valve system according to claim 52, wherein the latch is one of (i) pivotally connected to and (ii) integral with a first one of the proximal portion and the distal portion, and a second one of the proximal portion and the distal portion includes a recess configured to receive a portion of the latch.

54. The valve system according to claim 53, further comprising a line extending along a length of one of the proximal portion and distal portion connected to the latch configured to pivot the latch to disconnect the proximal and distal portions.

55. The valve system according to claim 54, wherein the line is slidable relative to the sheath and is pullable to pivot the latch out of the recess to disconnect the proximal portion and the distal portion.

56. The valve system according to claim 54, wherein the sheath includes at least one of (i) a servo and (ii) a motor adapted to pivot the latch between locked and unlocked positions, the line adapted to transmit an electric control signal to the at least one of (i) the servo and (ii) the motor.

57. The valve system of claim 47, wherein the sheath is adapted to split into the proximal portion and the distal portion at a predetermined location on the sheath upon application of at least one of (i) a predetermined force that pulls the proximal portion and the distal portion away from each other and (ii) a predetermined force that rotates the proximal portion and the distal portion relative to each other.

58. The valve system of claim 57, wherein the sheath includes a reduced wall thickness at the predetermined location.

59. The valve system of claim 57, wherein the sheath is partially cut at the predetermined location.

60. The valve system of claim 47, wherein the valve portion includes one or more flaps, each flap configured to form a pouch cavity which fills with blood when the valve is closed.

61. The valve of claim 1, wherein the valve portion includes one or more flaps, each flap configured to form a pouch cavity which fills with blood when the valve is closed.
